(19) United States
(12) Patent Application Publication
(10) Pub. No.: US 2024/0065603 A1
(43) Pub. Date: Feb. 29, 2024

(54) ELECTRIC GRIP GAUGE FOR ASSESSING HAND DEXTERITY

(71) Applicant: University of Utah Research Foundation, Salt Lake City, UT (US)

(72) Inventor: Jacob A. GEORGE, Salt Lake City, UT (US)

(21) Appl. No.: 18/117,316

(22) Filed: Mar. 3, 2023

Related U.S. Application Data
(60) Provisional application No. 63/401,543, filed on Aug. 26, 2022.

(51) Int. Cl. A61B 5/22

(52) U.S. Cl. CPC A61B 5/225; A61B 2560/0462; A61B 2562/06; G01R 33/072

(57) ABSTRACT

A grip gauge is configured to measure grip force from a single hand of a user. The grip gauge comprises a shell and a force sensor housed within the shell. The force sensor is configured to measure grip forces applied to the shell. The grip gauge also includes a control unit housed within the shell and communicatively connected to the force sensor, and a wireless transmitter communicatively connected to the control unit and configured to transmit measured grip forces to one or more external devices.

### CROSS-REFERENCE TO RELATED APPLICATIONS

This application claims the benefit of and priority to U.S. Provisional Patent Application No. 63/401,543, filed Aug. 26, 2022, which is incorporated herein by this reference in its entirety.

### STATEMENT REGARDING FEDERALLY SPONSORED RESEARCH

This invention was made with government support under OD029571 awarded by the National Institutes of Health. The government has certain rights in the invention.

### BACKGROUND

### Technical Field

This disclosure relates to devices and methods for measuring grip force. Such devices and methods can beneficially aid researchers and occupational therapists in assessing and/or treating patients with upper-limb dexterity deficiencies.

### Related Technology

Evaluating hand dexterity is a critical aspect of informing patient care for many neurodegenerative diseases and for assessing prosthetic technology. Current upper-limb dexterity assessments primarily target gross motor function and do not directly measure an individual's ability to finely regulate their grip force. For example, the Box and Blocks test (BBT) is a widely used test of upper-limb prosthesis function in which participants transfer small wooden blocks over a vertical barrier from one side of a box to another as quickly as possible in one minute. However, the BBT, like many other clinical measures of upper-limb function, does not directly measure the ability of an individual to finely regulate grip force, which is a vital aspect of hand control when manipulating fragile objects.

An increasingly popular test of fine motor function among researchers and occupational therapists is a fragile-object test, in which participants are instructed to lift and transfer an object while minimizing their applied grip force. Such tests ask the user to keep their grip force above the force required to pick up the object and below the force that would “break” the object. Instantiations of this fragile-object task have involved metal plates separated by a weak magnetic field, paper blocks held together loosely with toothpicks, and 3D-printed blocks held together weakly with embedded magnets.

However, these prior approaches have several shortcomings. For example, prior approaches are unable to effectively disentangle sensory and motor deficits of hand dexterity or effectively assess the role of sensory feedback in motor tasks. There is thus an ongoing need for improved electronic grip devices.

### SUMMARY

In one embodiment, a grip gauge is configured to measure grip force from a single hand of a user. The grip gauge comprises a shell and a force sensor housed within the shell. The force sensor is configured to measure grip forces applied to the shell. The grip gauge also includes a control unit housed within the shell and communicatively connected to the force sensor, and a wireless transmitter communicatively connected to the control unit and configured to transmit measured grip forces to one or more external devices.

The grip gauge can also include an accelerometer housed within the shell. The control unit is communicatively connected to the accelerometer and the wireless transmitter is configured to transmit measured accelerometer data to the one or more external devices.

The grip gauge can also include an indicator communicatively connected to the control unit. The control unit is configured to compare measured grip forces received from the force sensor to a predetermined threshold force and is configured to modulate the indicator based on the comparison. The indicator can comprise a light and/or a speaker disposed on the grip gauge and/or can be associated with an external device communicatively connected to the control unit. The control unit can be configured to modulate the indicator according to a discrete mode in which the indicator is in a first state when measured grip forces are below the threshold force and is in a second state when measured grip forces exceed the threshold force or to modulate the indicator according to a continuous mode in which the indicator is modulated according to how close measured grip forces are to the threshold force. For example, the control unit can control the volume of an audible sound generated by the speaker according to how close measured grip forces are to the threshold force.

The grip gauge can also include a Hall effect sensor housed within the shell. The control unit is communicatively connected to the Hall effect sensor and the wireless transmitter is configured to transmit measured Hall effect measurements to the one or more external devices. The Hall effect measurements can be made based using a platform with one or more magnetic surfaces, for example.

The grip gauge can be associated with a device configured to generate a display presenting one or more grip metrics. The grip metrics can include, for example: peak grip force; peak grip force during one or more transfers and/or one or more transfer subphases; variability in grip force across transfers and/or across transfer subphases; peak acceleration; peak acceleration during one or more transfers and/or one or more transfer subphases; variability in acceleration across transfers and/or transfer subphases; transfer speed; variability in transfer speed; transfer start/stop times; distance lifted off a platform; and/or relative location of placement on the platform.

The devices and methods described herein can beneficially improve the state of the art by, for example, providing a variety of assessment/training modalities for a fragile-object test, enabling real-time display of various metrics associated with measured grip force and device motion, and assisting in differentiating issues related to motor deficits from those related to sensory deficits.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A illustrates an embodiment of a grip gauge in use during a fragile-object test;

FIG. 1B illustrates the grip gauge in schematic form, showing communicative connection to an external device including a display;

FIGS. 2A and 2B illustrate a perspective view and exploded view, respectively, of an example embodiment of a grip gauge;

FIG. 3 illustrates an example display associated with an external device communicatively connected to the disclosed grip gauge; and

FIGS. 4A and 4B illustrate peak grip force and transfer time, respectively, during a fragile-object test using the disclosed grip gauge under conditions of no feedback, discrete feedback, and continuous feedback, for participants using an intact hand and an electromyography (EMG) controlled prosthesis.

### DETAILED DESCRIPTION

### Overview

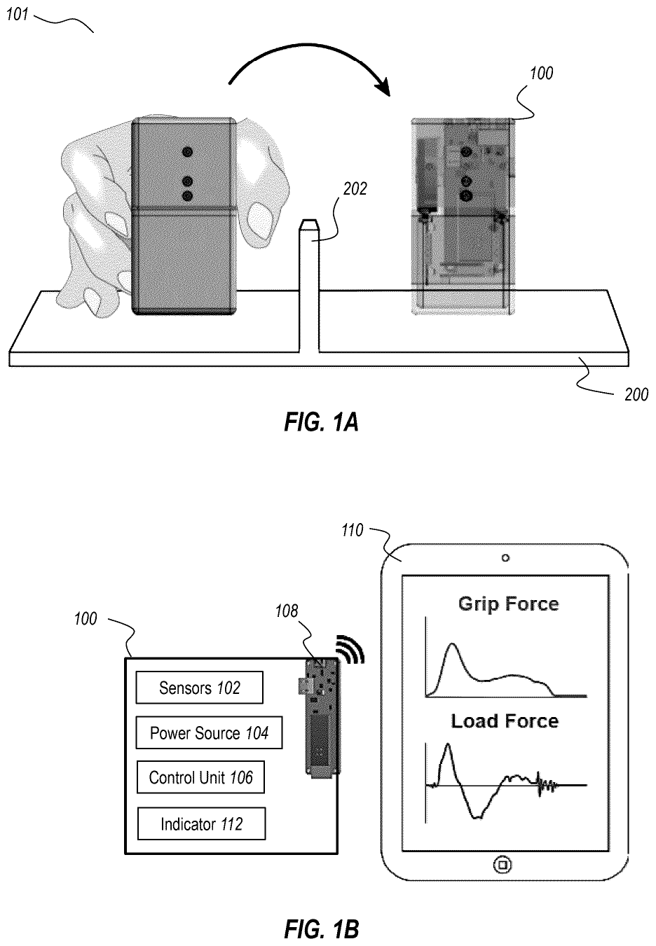

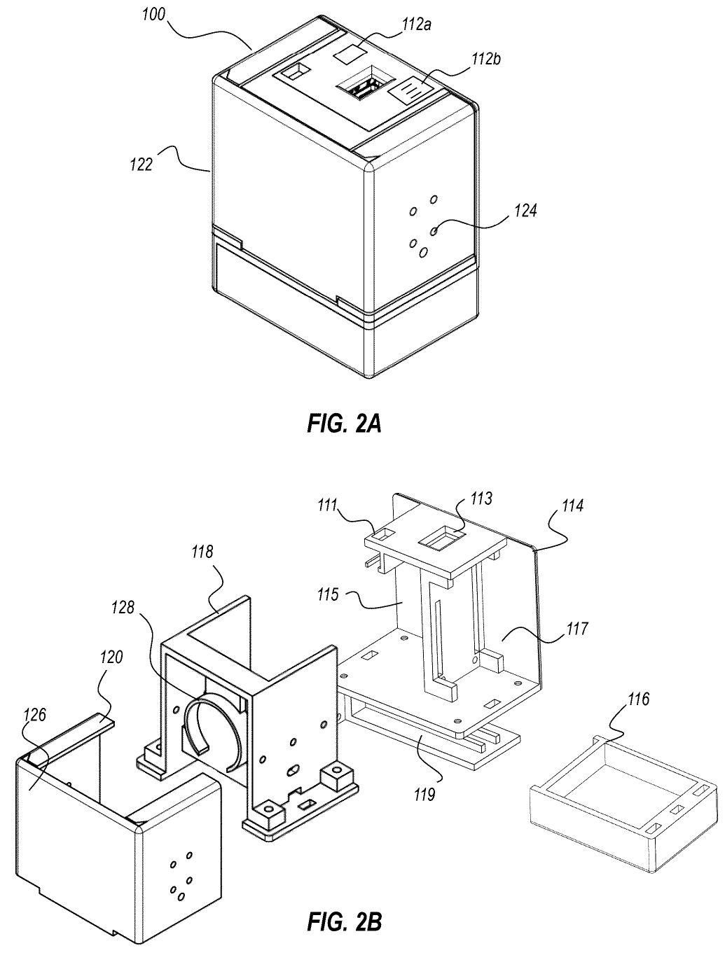

FIG. 1A illustrates an embodiment of a fragile-object test system 101, showing a grip gauge 100 in use during a fragile-object test. A typical fragile-object test involves introducing a “break” threshold to a block to replicate transferring a fragile object such as an egg. The user is asked to keep his/her grip force above the force required to pick up the object and below the force required to “break” the object.

In a typical implementation, the grip gauge 100 is placed on a platform 200 (or in a box or other suitable staging area) and the user is asked to repeatedly transfer the grip gauge 100 from one side of the platform 200 to the other over a vertical barrier 202 as quickly as possible during a timed period (e.g., one minute). The user's hand dexterity can be assessed based on the number of times the user is able to successfully transfer the grip gauge 100 from one side of the platform 200 to the other and based on the user's ability to minimize exceeding the threshold “break” force.

As used herein, a “trial” refers to a single fragile-object test carried out for a full test period (e.g., one minute). A “transfer” refers to a successful movement of the grip gauge 100 from a first predetermined section of the platform 200 to a second predetermined section of the platform 200, typically involving movement over the vertical barrier 202. Additional transfers are carried out by then moving the grip gauge 100 from the second section of the platform 200 back to the first section of the platform 200, and so on until the trial is complete. Within each transfer are multiple transfer subphases, including a grasping phase, loading phase, transfer phase, and release phase.

The grip gauge 100 described herein can be utilized to test intact/normal hands or to test prosthetic hands. The grip gauge 100 can be utilized to make clinical assessments regarding hand dexterity and/or can be utilized to provide hand dexterity training (e.g., as part of occupational therapy).

FIG. 1B illustrates the grip gauge 100 in schematic form. As shown, the grip gauge 100 can include a set of one or more sensors 102, a power source 104 (e.g., battery), a control unit 106, and a wireless transmitter 108. The one or more sensors 102 includes a force sensor (e.g., a load cell) configured to measure grip forces applied to the grip gauge 100. The set of sensors 102 can also include an accelerometer for measuring acceleration forces (also referred to herein as load forces) applied to the grip gauge 100.

In some embodiments, the set of sensors 102 includes a Hall effect sensor. The Hall effect sensor can be used to measure the position of the grip gauge 100 relative to the platform 200. For example, the platform 200 can include one or more magnetic surfaces that enable the Hall effect sensor to detect when the grip gauge 100 contacts the platform 200 and/or to determine the distance between the platform 200 and the grip gauge 100. This enables the grip gauge 100 to automatically count the number of transfers performed during a fragile-object test.

In some embodiments, the platform 200 includes at least two separate sections of opposite magnetic polarity, such as on either side of the vertical barrier 202. This allows the Hall effect sensor to detect which side of the vertical barrier 202 the grip gauge 100 is disposed and can ensure a counted transfer involves full movement from one side of the platform 200 to the other.

The control unit 106 is communicatively connected to the sensors 202 and to the wireless transmitter 108, and is configured to transmit measured grip forces (and/or other data obtained by the grip gauge 100) to one or more external devices 110. The external device 110 can generate a display presenting one or more grip metrics. Grip metrics may include, for example: peak grip force; peak grip force during each transfer and/or transfer subphase; variability in grip force across transfers and/or across transfer subphases; peak acceleration; peak acceleration during each transfer and/or transfer subphase; variability in acceleration across transfers and/or transfer subphases; transfer speed; variability in transfer speed; transfer start/stop times; distance lifted off a platform; and/or relative location of placement on the platform. The grip metrics may also include averages of any of the above metrics over N trials for a given user.

Various calculations related to the grip metrics may be calculated by the control unit 106, by the external device 110, or through a combination of both the control unit 106 and the external device 110. The underlying processing utilized to generate the grip metrics may therefore be divided between the control unit 106 and the external device 110 in a variety of ways.

The illustrated grip gauge 100 also includes an indicator 112 communicatively connected to the control unit 106. The control unit 106 can be configured to compare measured grip forces received from the force sensor to the selected threshold force and to modulate the indicator 112 based on the comparison. For example, the indicator 112 can include a light and/or speaker configured to change state based on whether the threshold force is met and/or based on how close the measured grip force is to the threshold force.

The grip gauge 100 can be operated under different modes/settings. In a “discrete mode,” the control unit 106 puts the indicator 112 in a first state when measured grip forces are below the threshold force and is in a second state when measured grip forces exceed the threshold force. For example, the grip gauge 100 may turn on a light and/or provide audible sound only when the measured grip forces exceed the threshold force. In a “continuous mode,” the control unit 106 modulates the indicator 112 according to how close measured grip forces are to the threshold force. For example, the brightness of a light and/or the volume of an audible sound may increase as gripping forces get closer to the threshold force.

The indicator 112 may additionally or alternatively be communicatively connected to other sensor types described herein, such as the accelerometer and/or Hall effect sensor. In some embodiments, the grip gauge 100 includes multiple indicators 112, each being associated with a different sensor. For example, a first indicator may be associated with the force sensor, a second indicator may be associated with the accelerometer, and/or a third indicator may be associated with the Hall effect sensor.

In the illustrated embodiment, the indicator 112 is disposed on the grip gauge 100. The functions of the indicator 112 may additionally or alternatively be provided by the connected external device 110. For example, the external device 110 can provide a visual and/or audio cue based on whether the threshold force is met and/or based on how close the measured grip force is to the threshold force.

The grip gauge 100 can beneficially be utilized to assist in differentiating dexterity issues based on sensory deficits from those based on motor deficits. For example, users may be tested in a fragile-object test using the grip gauge 100 in discrete mode (using only indirect tactile and sensory feedback) and in continuous mode (in which the indicator 112 is proportionate to applied grip force). Significantly better performance during continuous mode as compared to discrete mode indicates that sensory deficits likely play a significant role in the dexterity issues, whereas smaller differences or no difference in performance between the two modes indicates that sensory deficits play little or no role in the dexterity issues, suggesting that motor deficits play a more significant role.

The threshold force can be adjustable by the user. In some embodiments, the threshold force is automatically adjusted based on past user performance, with the intent to provide sufficient difficulty for the user while still remaining within the user's capabilities.

### Example Grip Gauge

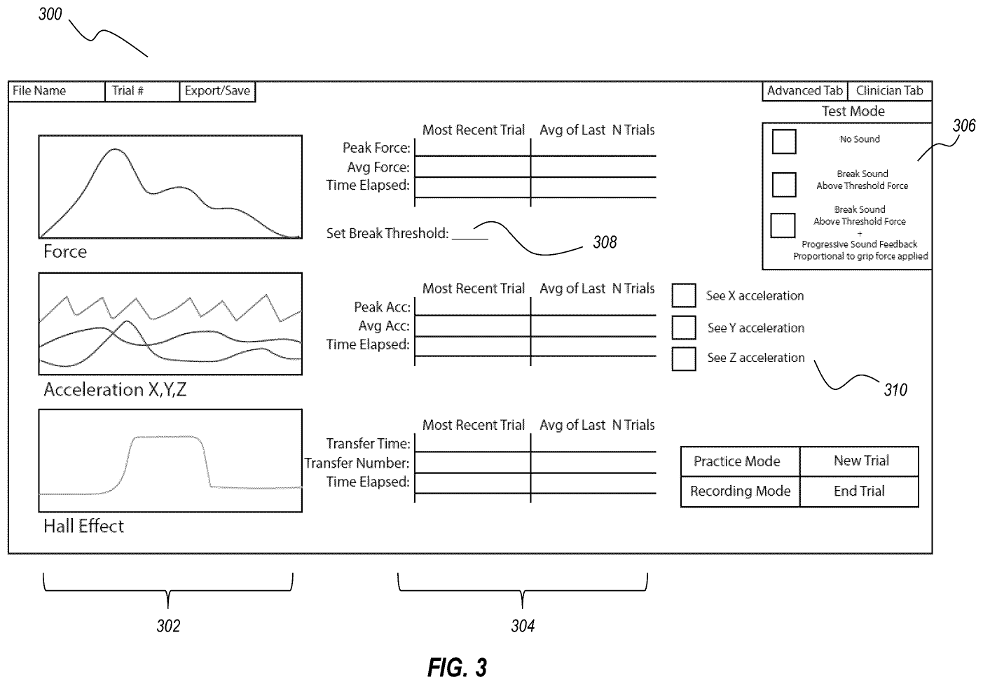

FIGS. 2A and 2B illustrate a perspective view and exploded view, respectively, of the example grip gauge 100. As shown, the grip gauge 100 can include a shell 122 within which the sensors, control unit, and wireless transmitter are housed. The shell 122 can include through holes 124 for passing linear guide rails associated with one or more sensors of the device. As discussed above, the grip gauge 100 can include an indicator in the form of a light 112a and/or speaker 112b.

As shown in FIG. 2B, the grip gauge 100 can include a housing piece 114 with a control unit section 115 and power source section 117 for supporting and housing such components. The housing piece 114 can also include a power switch access 111 and control unit port 113 for providing access to the control unit (e.g., access to a universal serial bus (USB) port of the control unit). The housing piece 114 can also include a base receiver 119 configured to receive and engage with the base piece 116. The base piece 116 includes a receptacle space that can be filled with a variable number of weights to adjust the overall weight of the grip gauge 100.

The grip gauge 100 can also include a core piece 118 configured to hold and position the force sensor and/or other sensors of the device. Aligning and connecting the housing piece 114 and the core piece 118 thereby brings the force sensor and/or other sensors into proper position with respect to the control unit.

A face plate 120 forms the remainder of the exterior of the shell 122 and provides an active surface 126 for transferring grip forces received from the user to the force sensor. For example, the force sensor may be held by mounting bracket 128 so that pressure applied against active surface 126 is transmitted to the force sensor. The internal side of the face plate 120 opposite the active surface 126 and facing the force sensor may include a protrusion to engage against the force sensor.

Other embodiments may include other construction forms. For example, some of the various pieces illustrated in FIG. 2B may be formed as an integral piece rather than separately connected, and/or the grip gauge 100 may be configured to place the various internal components in alternative positions.

### Example User Interface Display

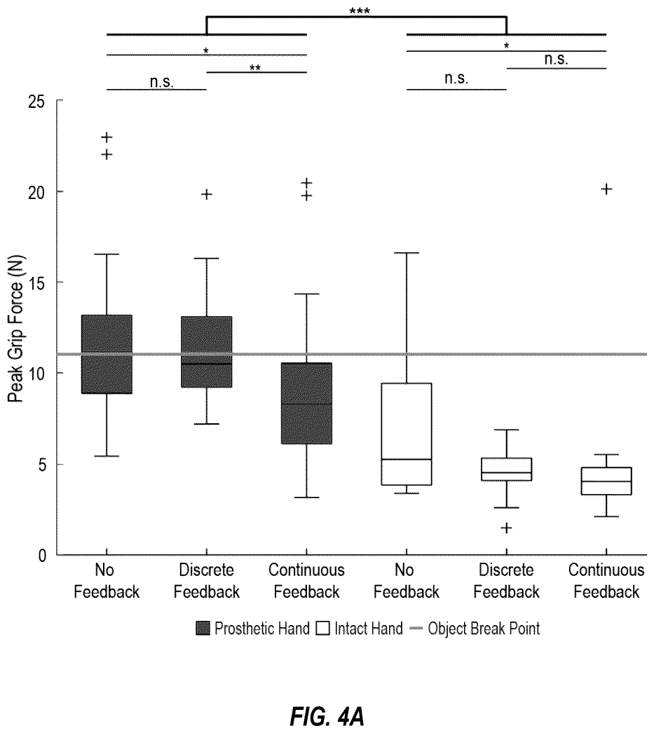

FIG. 3 illustrates an example display 300 that can be associated with an external device (e.g., external device 110) communicatively connected to the grip gauge 100. The display can present various grip metrics in real-time (i.e., with less than 3 s delay, less than 2 s delay, less than 1 s delay, less than 0.5 s delay, or essentially imperceptible delay) during a fragile-object test.

The display 300 can include a section 302 that graphically presents sensor data. Here, the section 302 shows grip force, acceleration (in X, Y, and Z axes) and Hall effect measurements (e.g., touches to the platform 200). The graphs can illustrate these respective measures over a particular trial or can be specific to a particular transfer within the trial. For example, the display 300 can be set to refresh the graphs after each transfer or after some set number of transfers, or can be set to show their respective measurements across the duration of the trial.

The display 300 can also include a section 304 that presents summary information related to the measured sensor data. Preferably, this summary information is aligned with the graphical data of section 302 such that, for example, summary information related to grip force is aligned with the graphical information related to grip force, summary information related to acceleration is aligned with the graphical information related to acceleration, and summary information related to Hall effect counts is aligned with the graphical information related to the Hall effect sensor.

As shown, the summary information can include peak measurements, average measurements, and associated time measurements. Other summary information may additionally or alternatively be displayed, including any combination of the various grip metrics described elsewhere herein. The summary information can be presented relative to each transfer (or subphases thereof), each trial, and/or over N number of trials.

The display 300 can also include one or more user selectable objects for adjusting aspects of the display 300, interfacing with the connected grip gauge 100, and/or managing the obtained data. For example, the display 300 can include a mode selection object 306 that enables selection of the operation mode of the grip gauge 100 (e.g., no feedback, discrete, or continuous). The display 300 can include a threshold force selection object 308 enabling adjustment of the threshold force at which a “break” is determined. The display 300 can also include a graph view object 310 enabling adjustments to the manner the data is presented. For example, object 310 enables the user to turn axes on and off with respect to illustrated acceleration data.

Other user selectable objects may also be included in the display 300. For example, objects may be included for zooming in/out of graphs, for adjusting the time period over which the graph data and/or summary information relates, and/or for selecting which metrics to display.

### Additional Control Unit & External Device Details

The control unit 106 and external device 110 can include one or more processors and computer-readable media such as computer memory stored on one or more hardware storage devices. The computer memory may store computer-executable instructions that when executed by one or more processors cause various functions to be performed, such as the acts recited herein. The control unit 106 and external device 110 may also be referred to herein as “computers” or “computer systems.”

Computer-readable media can include any media that can be accessed by a general purpose or special purpose computer system. Physical computer-readable storage media includes RAM, ROM, EEPROM, optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), and the like.

The control unit 106 and/or external device 110 may be interconnected to one or more other computing systems via one or more network connections. Network connections may include, but are not limited to, connections via wired or wireless Ethernet, cellular connections, or even computer to computer connections through serial, parallel, USB, or other connections. The control unit 106 and/or external device 110 may be included in a distributed system environment in which local and remote computer systems, which are linked (either by hardwired data links, wireless data links, or by a combination of hardwired and wireless data links) through a network, both perform tasks. In a distributed system environment, program modules may be located in both local and remote memory storage devices.

The control unit 106 and/or external device 110 may have input hardware and software user interfaces to facilitate user interaction. For example, the control unit 106 and/or external device 110 may be configured to operate with a keyboard, mouse, touchpad, touchscreen, camera, manual actuator (e.g., buttons, switches, dials) for allowing a user to input data into the controller. In addition, various software user interfaces may be available. Examples of software user interfaces include graphical user interfaces, text command line-based user interface, function key or hot key user interfaces, and the like.

### Working Example

A grip gauge as disclosed herein was constructed with a 100 pound load cell, triple-axis accelerometer, and control unit with wireless transmitter housed within a polymer shell. The shell base was filled with lead weights to vary the weight of the device from 132 to 414 g. The assembled device measured 60.5×42.7×80.5 mm.

Three test conditions or modes were utilized. In the first test condition, participants simply pick up and transfer the device as many times as possible within one minute while grip force is recorded. This test condition provides a measure of the transfer rate, similar to the BBT, and the users implicit grasping force, similar to the Grasping Relative Index of Performance.

In the second test condition, an upper threshold on the grip force is established such that the device will “break” and emit an audible sound if the participant's grip force exceeds the threshold. The participants are instructed to pick up and transfer the device as quickly as possible without breaking it. The difficulty of the task can be adjusted by lowering the break threshold or by increasing the weight of the device. In this study, the breakpoint was set to 11.2 N and the device weighed 414 g. The ratio of break force to weight in this study was 0.027 N/g. Because the device does not visually deform with increasing grip pressure, the only feedback the participants have regarding their applied grip force is endogenous (e.g., proprioception from forearm muscles or efference copy) or indirect (e.g., sounds of the prosthesis motor). Thus, this test condition provides a measure of the participant's innate sensorimotor grasping precision.

In the third test condition, the participant is instructed to pick up and transfer the device as quickly as possible without breaking it. However, in this test condition, a tone is played continuously, and the pitch of the tone increases as the applied grip force approaches the break threshold. If the break threshold is exceeded, a second tone is played indicating the device has broken. Using this test condition (continuous feedback) in conjunction with the previous test condition (discrete feedback) provides a way to systematically probe the impact of tactile sensory feedback on grasping precision. Significantly greater performance with continuous auditory feedback implies tactile/sensory feedback is impaired.

Three neurologically healthy and physiologically intact participants volunteered in this study. Participants were between the ages of 18 and 21 (100% male). Participants were instructed to complete the three aforementioned test conditions ten times each, using both their intact hand and an EMG-controlled prosthesis. Participants moved the device over a 2-in vertical barrier.

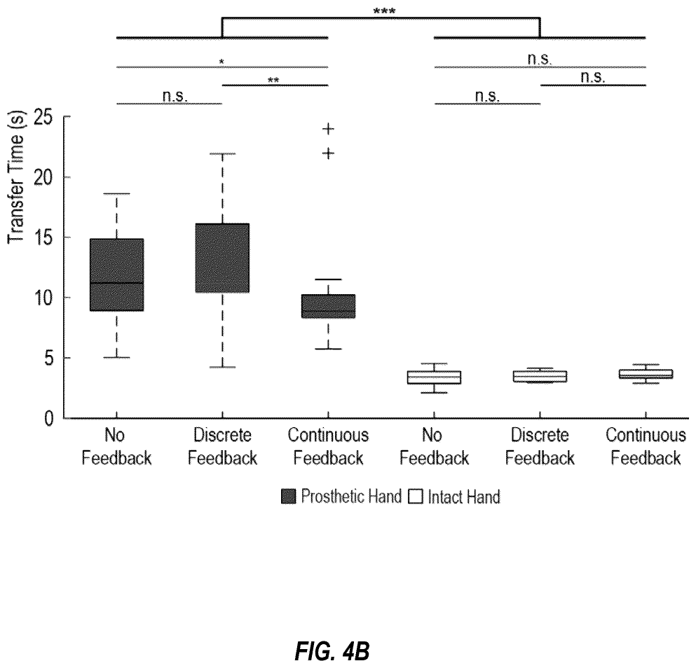

Results are illustrated in FIGS. 4A and 4B, which show peak grip force and transfer time, respectively, under conditions of no feedback, discrete feedback, and continuous feedback. Overall, grip force (FIG. 4A) and transfer time (FIG. 4B) were significantly greater for the prosthetic hand compared to that of the intact hand (p's<0.05; Wilcoxon rank-sum test). Across all three test conditions, average transfer time was 283.3% longer for the prosthesis and grip force was 92.6% stronger for the prosthesis.

When a break threshold was introduced (discrete feedback), we observed no significant difference in performance for the prosthetic hand or intact hand. For the prosthetic hand, this resulted in a substantial failure rate (i.e., roughly 50% of the peak forces exceed the blue line representing the break threshold in FIG. 4A). For the intact hand, implicit grasping force (no feedback condition) was already below the break threshold and remained similar.

When continuous auditory feedback was introduced on top of the break threshold, prosthetic performance improved significantly (p<0.05; Wilcoxon rank-sum test). That is, transfer time was reduced by 20.3% and grasping force was reduced by 23.7%.

For the intact hand, continuous auditory feedback did not significantly improve the task performance relative to the discrete feedback condition. Overall, the median transfer time and grip force of the intact hand was similar across all three test conditions.

The results show that a lack of tactile sensory feedback is a significant reason for poor prosthetic control, as evidenced by improved dexterity when continuous auditory feedback is provided in proportion to exerted grip force.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein can omit components that are not specifically described. For example, sensor types or measured metrics not specifically described herein may optionally be omitted.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A grip gauge configured to measure grip force from a single hand of a user, the grip gauge comprising:
a shell;
a force sensor housed within the shell, the force sensor being configured to measure grip forces applied to the shell;
a control unit housed within the shell and communicatively connected to the force sensor; and
a wireless transmitter communicatively connected to the control unit and configured to transmit measured grip forces to one or more external devices.

2. The grip gauge of claim 1, wherein the force sensor is a load cell.

3. The grip gauge of claim 1, further comprising an accelerometer housed within the shell, wherein the control unit is communicatively connected to the accelerometer and wherein the wireless transmitter is further configured to transmit measured accelerometer data to the one or more external devices.

4. The grip gauge of claim 1, further comprising an indicator communicatively connected to the control unit, wherein the control unit is configured to compare measured grip forces received from the force sensor to a predetermined threshold force and is configured to modulate the indicator based on the comparison.

5. The grip gauge of claim 4, wherein the indicator comprises a light and/or a speaker disposed on the grip gauge.

6. The grip gauge of claim 4, wherein the indicator is associated with an external device communicatively connected to the control unit.

7. The grip gauge of claim 4, wherein the control unit is configured to modulate the indicator according to a discrete mode in which the indicator is in a first state when measured grip forces are below the threshold force and is in a second state when measured grip forces exceed the threshold force.

8. The grip gauge of claim 4, wherein the control unit is configured to modulate the indicator according to a continuous mode in which the indicator is modulated according to how close measured grip forces are to the threshold force.

9. The grip gauge of claim 7, wherein the indicator comprises a speaker and wherein the control unit controls the volume of an audible sound generated by the speaker according to how close measured grip forces are to the threshold force.

10. The grip gauge of claim 1, further comprising a Hall effect sensor housed within the shell, wherein the control unit is communicatively connected to the Hall effect sensor and wherein the wireless transmitter is further configured to transmit measured Hall effect measurements to the one or more external devices.

11. The grip gauge of claim 1, wherein the shell comprises a base portion configured to house one or more weights.

12. The grip gauge of claim 1, wherein the shell is formed in a block shape.

13. A grip gauge system, comprising:
the grip gauge of claim 1; and
an external device communicatively connected to the electronic grip gauge and configured to receive data from the wireless transmitter, the external device comprising one or more processors and one or more hardware storage devices that store instructions that are executable by the one or more processors to cause the external device to generate a display presenting one or more grip metrics.

14. The grip gauge system of claim 12, wherein the one or more grip metrics comprise: peak grip force; peak grip force during one or more transfers and/or one or more transfer subphases; variability in grip force across transfers and/or across transfer subphases; peak acceleration; peak acceleration during one or more transfers and/or one or more transfer subphases; variability in acceleration across transfers and/or transfer subphases; transfer speed; variability in transfer speed; transfer start/stop times; distance lifted off a platform; and/or relative location of placement on the platform.

15. The grip gauge system of claim 13, wherein the one or more grip metrics comprise: peak grip force during each transfer and/or each transfer subphase; and/or peak acceleration during each transfer and/or each transfer subphase.

16. A fragile-object test system, comprising:
the electronic grip gauge of claim 1; and
a platform configured for placement of the electronic grip gauge thereon.

17. The fragile-object test system of claim 15, wherein the platform further comprises one or more magnetic surfaces and the electronic grip gauge further comprises a Hall effect sensor housed within the shell, wherein the control unit is communicatively connected to the Hall effect sensor and wherein the wireless transmitter is further configured to transmit grip gauge position measurements to the one or more external devices.

18. The fragile-object test system of claim 16, wherein the platform comprises at least two separate sections of opposite magnetic polarity, the platform optionally comprising a vertical barrier dividing the at least two separate sections.

19. A method for assessing hand dexterity of a user to distinguish sensory deficits and motor deficits, the method comprising:
providing a grip gauge as in claim 4;
operating the grip gauge, during a fragile-object test, in a discrete mode in which the indicator is in a first state when measured grip forces are below the threshold force and is in a second state when measured grip forces exceed the threshold force;
operating the grip gauge, during a fragile-object test, in a continuous mode in which the indicator is modulated according to how close measured grip forces are to the threshold force;
if user performance during the continuous mode is improved relative to during the discrete mode by at least a predetermined level, determining that hand dexterity deficits are primarily due to sensory deficits.

20. The method of claim 19, wherein the user utilizes a hand prosthesis to carry out the fragile-object test in discrete mode and continuous mode.
